(19) United States
(12) Patent Application Publication
(10) Pub. No.: US 2014/0171852 A1
(43) Pub. Date: Jun. 19, 2014

(54) TROCAR AND TROCAR-NEEDLE INTEGRATED SYSTEM FOR GLAUCOMA DRAINAGE SURGERY

(71) Applicant: Ophthalmic Implants Private Limited, Singapore (SG)

(72) Inventor: Eugene Khor, Victoria (CA)

(73) Assignee: Ophthalmic Implants Private Limited, Singapore (SG)

(21) Appl. No.: 14/103,166

(22) Filed: Dec. 11, 2013

Related U.S. Application Data
(60) Provisional application No. 61/739,583, filed on Dec. 19, 2012.

(30) Foreign Application Priority Data
Apr. 12, 2013 (AU) 2013 204 285

(51) Int. Cl. A61F 9/007; A61B 17/34

(52) U.S. Cl. CPC A61F 9/00781; A61B 17/3415
USPC 604/8; 604/164.01

(57) ABSTRACT

A trocar comprising a hollow barrel having an open side extending along the hollow barrel, the hollow barrel terminating at a first end with a pointed tip for incision of the trocar through tissue and having a needle connecting part. The open side may extend through the needle connecting part. The needle connecting part may be a collar. The trocar is used in combination with a needle. A method of use of the trocar is disclosed.

### CROSS-REFERENCE TO RELATED APPLICATIONS

This application claims the benefit of 35 USC 119(e) in respect of U.S. provisional patent application No. 61/739,583 filed Dec. 19, 2012.

### FIELD

The field of the invention is in Ophthalmology, in particular, as it pertains to surgical intervention using the glaucoma drainage device (GDD), also known as the glaucoma-filtering device, in the treatment of glaucoma.

### BACKGROUND

The GDD is a surgical implant that permits the drainage of excess fluid from the interior eye to the exterior, thereby lowering ocular pressure, the principal indication for glaucoma. The GDD has been around since the late 1970s. GDDs in use today have comprises a length of small diameter tubing attached to a base plate. The tube is inserted to the anterior chamber of the eye and fluid drains out to be dissipated through the plate. 2 standard designs, the Ahmed and the Baerveldt GDDs are the most popular in use today.

Glaucoma is a clinical condition where the pressure in the eye (ocular pressure) is elevated. Left untreated, this elevated ocular pressure can lead to blindness. Glaucoma filtering surgery is one of several treatment methods that have been developed to control and moderate ocular pressure to control the progression of glaucoma by creating an alternative outflow pathway for aqueous fluid to leave the eye. In glaucoma filtering surgery, an implant device termed the glaucoma drainage device (GDD) is inserted into the patient's eye. GDD implant devices have a long history dating back to 1912. Contemporary GDD devices introduced since 1979 typically comprise a tube that is connected to a plate. The open tube end is placed in the anterior chamber of the eye while the plate is located under the conjunctiva. Excess fluid drains out from the anterior chamber via the tube where it is dissipated through the plate, thereby reducing excess intraocular pressure (IOP).

When utilizing the Ahmed and the Baerveldt GDDs the body of the devices is first secured to the conjunctiva/sclera region of the eyeball. The tube end that extends over the anterior chamber is trimmed, normally after the incision is made, to a length that permits insertion into the anterior chamber by approximately 2-5 mm. The incision of the anterior chamber is normally made with a 23-gauged (0.6414 mm outer diameter) needle that is withdrawn immediately after the cut is made. This makes locating the incision site another factor during surgery.

### SUMMARY

There is disclosed a trocar comprising a hollow barrel having an open side extending along the hollow barrel, the hollow barrel terminating at a first end with a tip, which may be rounded (smooth-edged point), for incision of the trocar through tissue. In an embodiment, the trocar has a needle connecting part. The open side may extend through the needle connecting part. The needle connecting part may be a collar. The trocar is used as a standalone or in combination with a needle. Methods of use of the trocar are disclosed.

A trocar that is semi-circular in shape with two distinct ends, the first end has a tapered edge and the opposite end has a collar. The device is placed at the incision site during glaucoma drainage surgery to facilitate the identification of the incision site and the guidance and placement of the tube end of a glaucoma drainage device.

An integrated delivery system that combines a needle in the form of a needlepoint device having a barrel incorporating a trocar and is used for making an incision on the eye surface that subsequently permits the trocar to be placed at the incision site by separating the trocar from the barrel of the needlepoint device.

A combination needlepoint device incorporating a trocar used for making an incision on the eye surface with a trocar self-release mechanism that subsequently permits the trocar to be placed at the incision site by single-handed release of the trocar from the barrel of the needlepoint device by activating the self-release mechanism.

A trocar and needle assembly is disclosed having a needle having a handle, a trocar having a hollow barrel with an open side extending along the hollow barrel, the hollow barrel terminating at a first end with a tip for placement of the trocar through tissue; and the handle having a connecting part for holding the trocar around the needle.

### BRIEF DESCRIPTION OF THE FIGURES

There will now be described embodiments of the trocar with reference to the Figures by way of example, in which like reference characters denote like elements and in which:

FIGS. 1A, 1B and 1C respectively show a top view, side view and cross-section of an embodiment of a trocar. The trocar has a length of a half cut-off tube, ending at one end (distal) that has a tapered edge that is non-cutting. At the other end (proximal), the trocar terminates in a collar.

FIGS. 1D-1G show a second embodiment of a trocar, in top view, side view, section and perspective respectively.

FIGS. 1H-1K show a third embodiment of a trocar, in top view, side view, section and perspective respectively

The trocar can also have a proximal end where the collar is replaced by a stop that prevents the trocar from sliding into the eye (FIG. 1E & I).

FIG. 2A and 2B respectively shows a top view of a trocar integrated into a two-piece delivery system incorporating a needle and a section through FIG. 2A along the line AA. The needle has a needlepoint that makes the incision. The needle fits into the barrel of the trocar.

FIG. 3, together with FIG. 3A, which is a section along the line BB in FIG. 3, and FIG. 3B showing a needle and trocar separated, show the trocar of FIGS. 1A-1C integrated into a two-piece delivery system incorporating a needle that has an added trocar self-release mechanism facilitating the placement of the trocar immediately after the incision is made with the single-handed action.

FIGS. 4A-4D shows a series of steps taken to insert a drainage tube including (a) placement of the trocar on the eye, (b) placement of the tube in the trocar ready for insertion of the tube, (c) turning of the trocar to remove the trocar, (d) and removing the trocar leaving the tube in place.

### DETAILED DESCRIPTION

The disclosed trocar is used for glaucoma drainage device surgery, and is preferably used within a trocar-needle integrated delivery system but can also be used as a standalone tool. Detailed descriptions of the preferred embodiment of the trocar and trocar-needle integrated delivery system are provided herein. It is to be understood, however, that the present invention may be embodied in various other similar forms. Therefore, specific details disclosed herein are not to be interpreted as limiting, but rather as a basis for the claims and as a representative basis for teaching one skilled in the art to employ the present invention in virtually any appropriately detailed design, system, structure or manner. While the invention has been described in connection with a preferred embodiment, it is not intended to limit the scope of the invention to the particular form set forth, but on the contrary, it is intended to cover such alternatives, modifications, and equivalents as may be included within the appended claims.

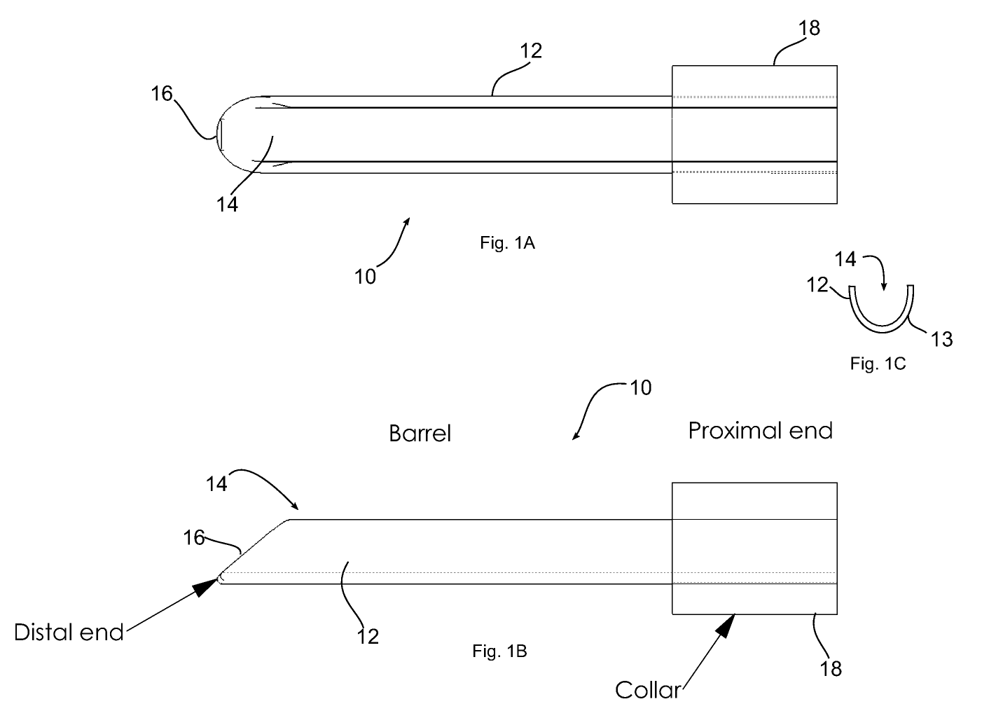

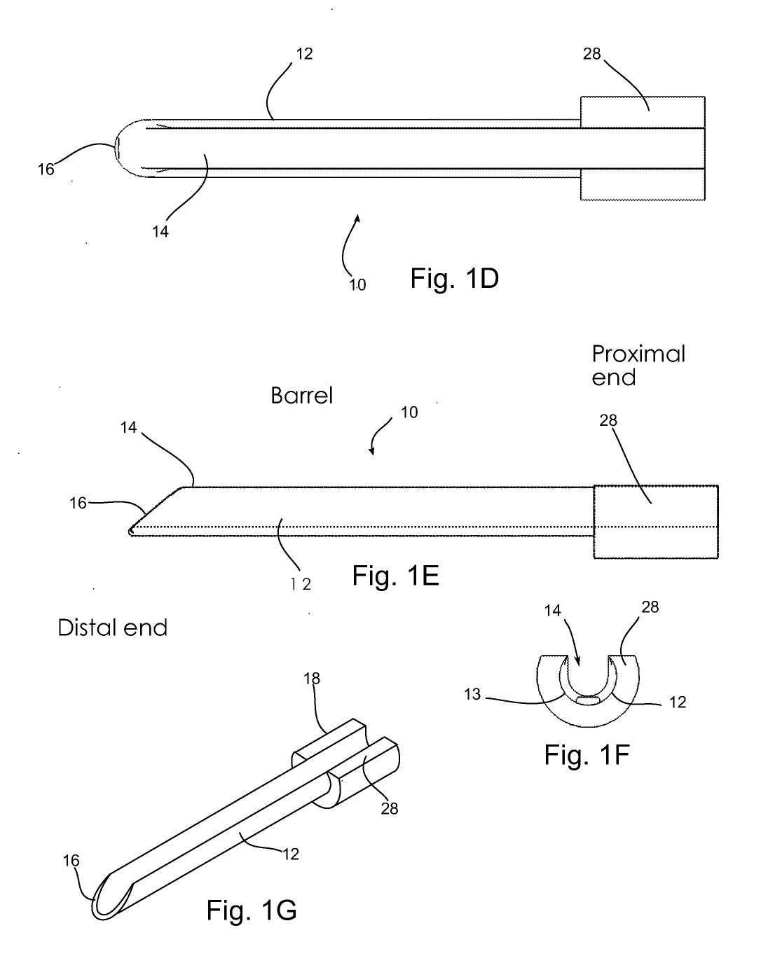

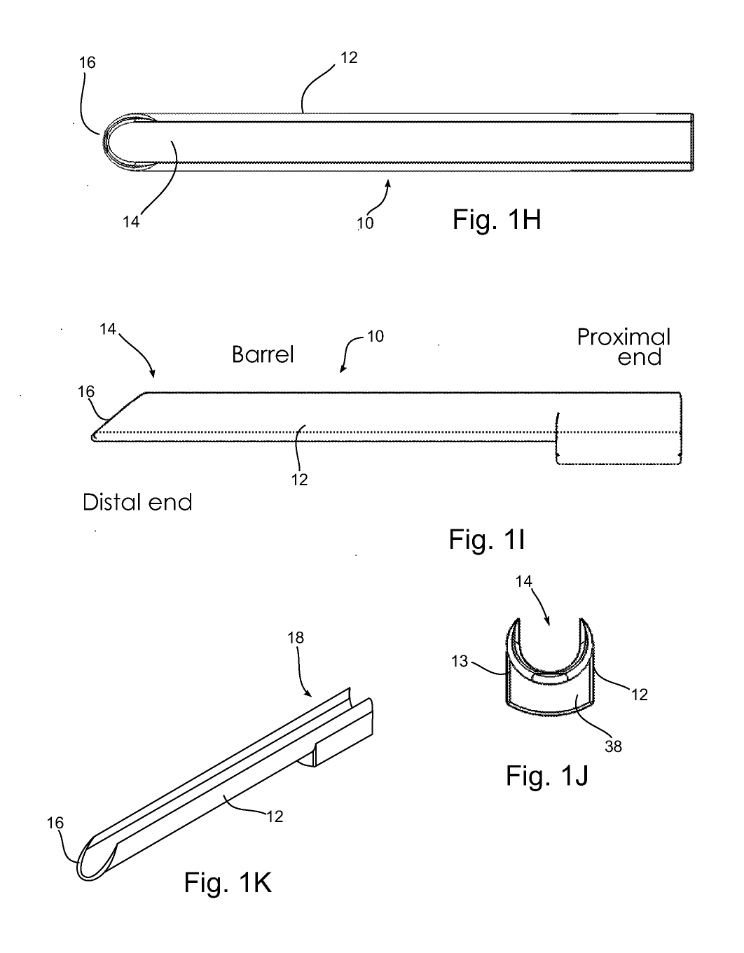

Referring to FIGS. 1A, 1B and 1C in particular, a trocar 10 is semi-circular in shape to facilitate the guidance and placement of a GDD tube as well as permit later withdrawal of the trocar 10. Retrieval is facilitated by turning clockwise or counterclockwise, depending on the orientation of the trocar 10, and pulling up thereby retrieving the trocar 10 while leaving the tube of the GDD in place at the surgical site. The material that can be utilized to fabricate the trocar 10 are metals for example but not limited to stainless steel (preferably of medical grade); ceramics such as but not limited to alumina, silicon-carbide, and silicon-nitride (all preferably of medical grades); and plastics or polymers, preferably but not limited to rigid forms, for example but not limited to Teflon, Delrin (acetal) and PEEK (all preferably of medical grades). Any other combination of materials that can be shaped similarly can also be used.

As shown in FIGS. 1A, 1B and 1C trocar 10 comprises a hollow barrel 12 having an open side 14 extending along the hollow barrel 12, the hollow barrel terminating at a first end with a rounded and non-cutting tip 16 for incision of the trocar through tissue. In some embodiments, the tip 16 may be pointed. The trocar 10 may have a needle connecting part 18. The open side 14 may extend through the needle connecting part. The needle connecting part 18 may be a collar. The trocar 10 is used in combination with a needle. In some embodiments, the needle may be designed to receive a trocar 10 without a collar 18 and the end of the trocar 10 away from the pointed tip 16 may be formed in any suitable manner to be received within a needle. If the needle has a slot shaped like the end of the trocar 10, the trocar 10 may simply be press fit or otherwise removably secured in the needle, as by small ridges or grooves on the trocar 10 that cooperate with corresponding ridges or grooves in the slot in the needle. Removably in this context means that the needle and trocar are connected together when an incision is made to access the anterior eye chamber, but can be easily separated so as to permit the trocar to remain at the site of the incision while the needle is removed by a surgeon using gentle forces that do not cause a risk of injury to a patient.

The length of the trocar is essentially a half cut-off tube, forming a trough with base 13. The length of the half cut-off tube may for example be between 0.5 mm to 50 mm. At the distal end, the trocar has a tapered edge 16 that is non-cutting. This is the end that projects into the anterior chamber at the incision site. At the proximal end, the trocar terminates in a collar 18 or other suitable needle connecting part. The collar 18 is so designed to act as a stop, preventing the trocar from accidentally slipping into the anterior chamber of the eye. The collar can be replaced by a stop, essentially a protrusion on the lower end of the half-cut tube at the distal end. This stop acts as a safety point to prevent the trocar from easily slipping into the eye. The half cut-off tube provides a channel for the tube end of the GDD to slip in, thereby facilitating easier guidance of the tube end of the GDD into the anterior chamber of the eye.

FIGS. 1D-1G show an embodiment of a trocar in which the needle connecting part 28 is flattened, to be level with the top of the barrel of the trocar 10, but otherwise extends around the barrel of the trocar 10. FIGS. 1H-1K show an embodiment of a trocar 10 in which the needle connecting part 38 is on the base 13 of the trocar 10, but does not extend outward laterally more than the body of the trocar 10. The needle connecting part 38 here forms a stop.

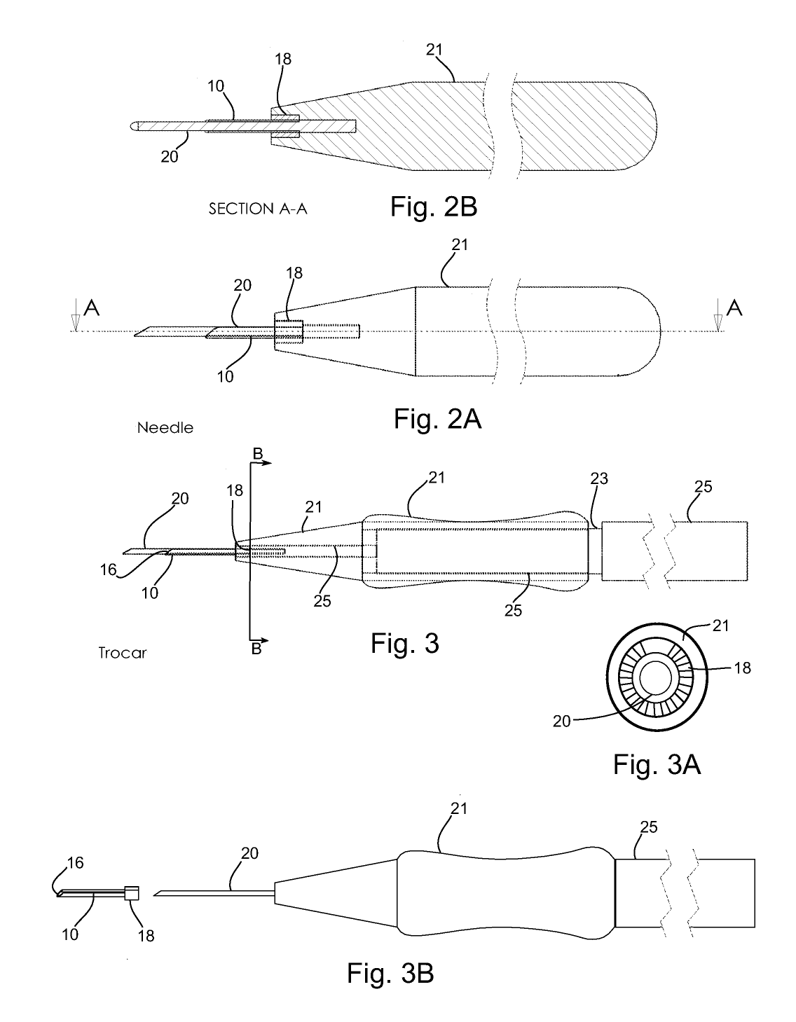

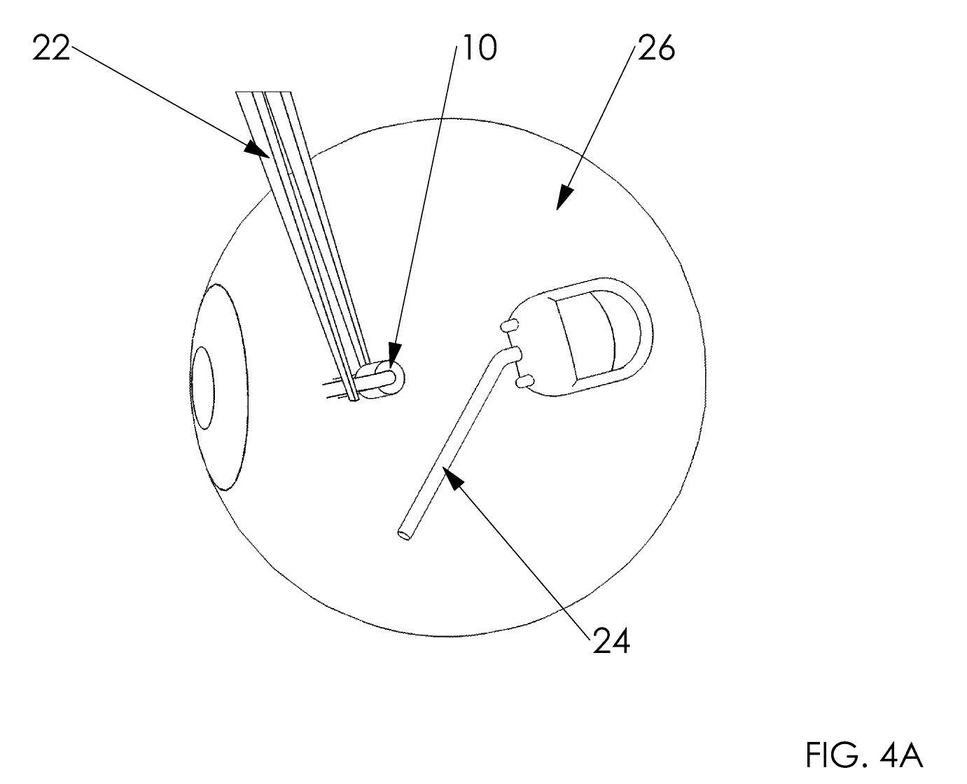

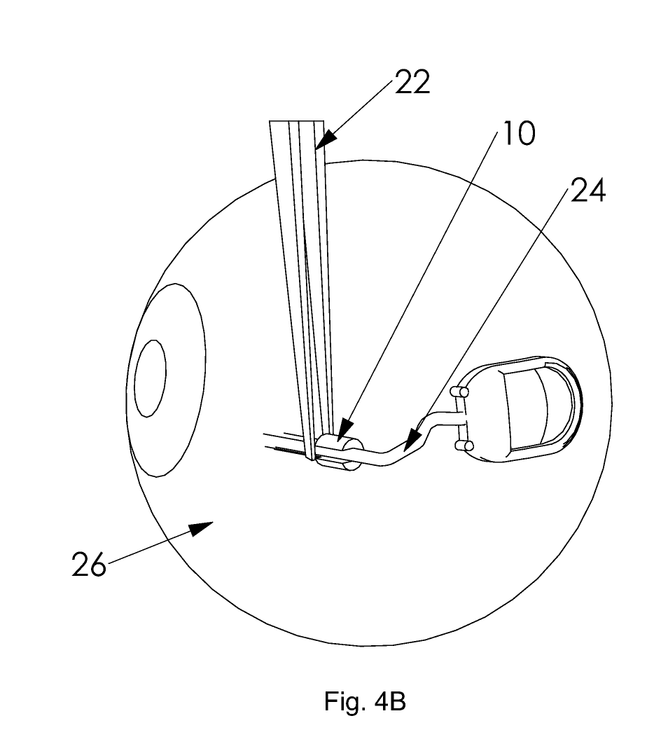

In the integrated configuration, the tool is conceived as two integral but separate components comprising a needle 20 and the trocar 10, as seen for example in FIGS. 2A, 2B and 3. The needle 20 in an embodiment is a 23-gauge point cylindrical shaft of between 0.50 mm to 100 mm in length. The forward end is shaped as a needlepoint. The other end of the needle 20 resides in the handle 21. The handle 21 has a slot or enlarged opening that accommodates the needle connecting part 18 of the trocar 10. In other embodiments, the handle 21 may have a protrusion or protrusions that fit into corresponding slots or holes on the trocar 10. The slots or protrusions on the handle 21 form connecting parts for holding the trocar 10 around the needle 20. The material that can be utilized to fabricate the shaft and the handle 21 are metals for example but not limited to stainless steel (preferably of medical grade); ceramics such as but not limited to alumina, silicon-carbide, and silicon-nitride (all preferably of medical grades); and plastics or polymers, preferably but not limited to rigid forms, for example but not limited to Teflon, Delrin (acetal) and PEEK (all preferably of medical grades). Any other combination of materials that can be shaped similarly can also be used.

The integrated delivery system is diagrammatically depicted in FIGS. 2A, 2B and 3. The needle's 23-gauge shaft is designed as a needle assembly that fits into a lumen of the semi-circular trocar or “tube-guide”. In this configuration the needlepoint 20 is withdrawn immediately after the incision is achieved by separating the trocar from the needle cum handle component, where the non-cutting edge of the trocar 16 extends into the incision keeping the incision site accessible as well as reducing the possibility of inadvertent damage to the site. Removal is by the surgeon holding the trocar 10 with a pair of forceps or tweezers while pulling the needle assembly 20 out. The fit of trocar 10 is firm enough to ensure it remains in place when the incision is made but not tight enough that the trocar cannot be removed during separation and placement by the surgeon.

The integrated delivery system with self-release mechanism is diagrammatically depicted in FIG. 3, 3A and 3B. In the self-release configuration, the needle 20 is fixed to a handle part 25 and on the handle part 25 is a sliding, forward, handle 21 and handle part 23-25 at the rear. The sliding part 21 receives the collar 18 or other needle connecting part in a recess or opening in the sliding part 21. The surgeon activates the single-handed release mechanism gently to place the trocar 10 at the incision site. The surgeon holds the handle 21 at the front and slides it backwards until the forward handle 21 closes the gap 23. This action separates the trocar collar 10 from the handle 21. Further sliding back of the handle separates the needle 20 cum handle 21 from the trocar 10 leaving the trocar 10 in place at the incision site. In FIG. 3B, the needle 21 and handle 20 are in the slid back position. In the position shown in FIG. 3, the trocar 10 is held in part 20 by friction. As the needle 20 and handle part 21 are moved back on the handle 25, the trocar 10 is pushed out of (disengaged from) the needle 20 and handle part 21 and is then free to slide along the needle 20. The surgeon can at this point hold the trocar with tweezers as the needle 20 is withdrawn from the eye leaving the trocar inserted ready to facilitate devise being fed into the eye. The surgeon may utilize a pair of forceps or tweezers during the release to hold the trocar to ensure the trocar placement is appropriate.

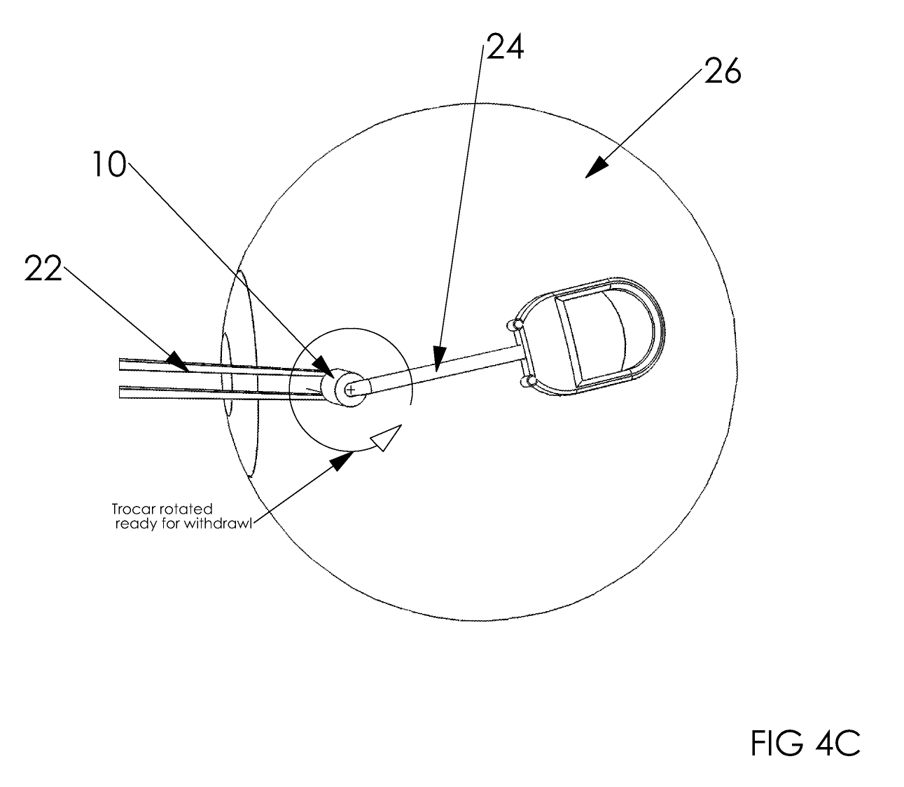

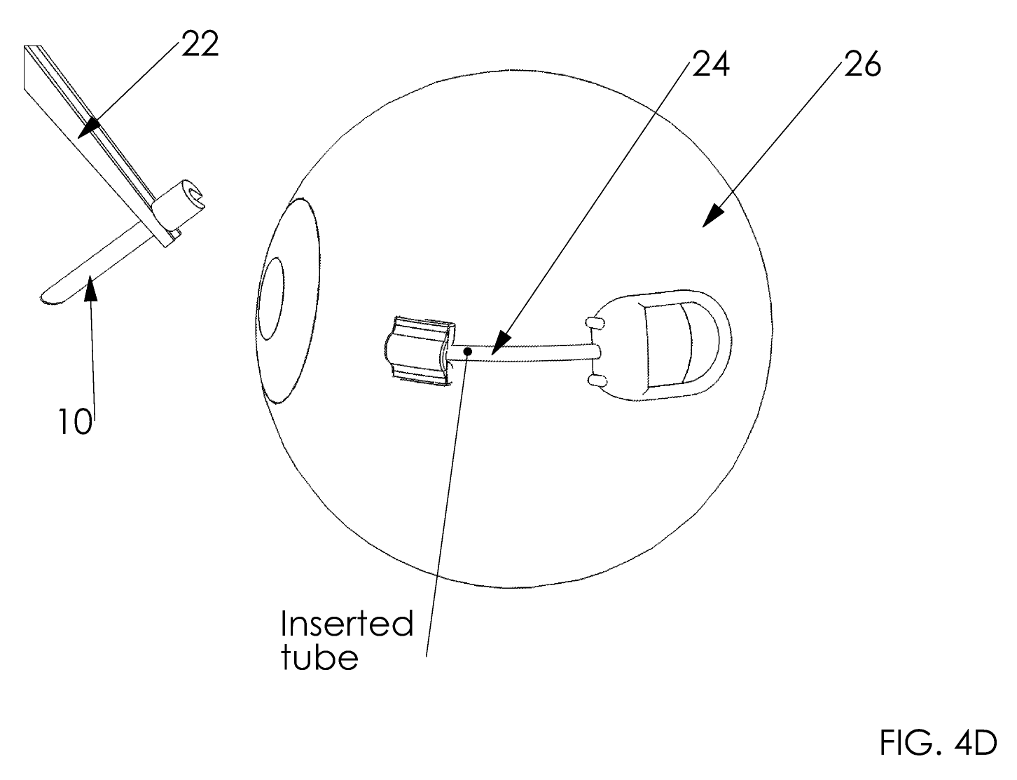

FIGS. 4A-4D show how to insert a tube with the trocar 10 and remove the trocar 10, leaving the GDD tube behind. In the method, the steps comprise: placing a trocar 10 on an eye 26 using forceps 22, where the trocar 10 has a trough shape and a base with the trough opening in a direction (away from the eye 26) that allows access for insertion of a drainage tube 24; placing the drainage tube 24 in the trocar 10 ready for insertion of the tube 24; inserting the tube 24 into the eye 26; turning the trocar 10 with forceps 22 to allow removal of the trocar 10; and removing the trocar 10 with forceps 22 leaving the tube 24 in place on the eye 26. In the turning step of FIG. 4C, the trocar 10 is preferably turned so that the opening of the trough is towards the eye 26.

Embodiments of the disclosed assembly facilitate the guidance of the GDD tube that extends from the body of the Ahmed and the Baerveldt GDDs. The disclosed assembly can also be used to guide small tubes of other designs of GDD implants such as the Molteno, used for glaucoma drainage surgery. More particularly, the disclosed assembly presents a designed accessory called a trocar that permits in various embodiments:

Guiding and placement of the tube end of the Ahmed and the Baerveldt GDDs into the anterior chamber of the eye at the incision site.

Guiding and placement of the tube from other designs of implants used for glaucoma drainage surgery.

The trocar can also be combined with a delivery system that incorporates the trocar with a needle. The needle is use to make the incision on the eye and the incorporated trocar is separated from the needle at the incision site to facilitate placement of the GDD tube at the incision site.

The trocar as a standalone or as a combination delivery system may offer the following benefits: (1) Reduce the time taken to re-locate the incision site after the initial incision is made. (2) Introduce a straightforward and error-free method to direct the tube into the anterior chamber of the eye. (3) Permit the surgeon a measure of convenience and confidence of ensuring a proper placement of the tube thereby improving the overall surgical procedure of glaucoma drainage surgery. (4) The invention will facilitate easier training of ophthalmologists in the GDD procedure increasing the better treatment of glaucoma.

This improves the surgical procedure of glaucoma drainage surgery using the Ahmed and the Baerveldt GDDs and other tube containing GDDs by permitting ready and precise siting of the tube end at the incision site.

What is claimed is:

1. A trocar comprising;
a hollow barrel having an open side extending along the hollow barrel, the hollow barrel terminating at a first end with a tip for placement of the trocar through tissue.

2. The trocar of claim 1 in which the tip is rounded.

3. The trocar of claim 1 having a needle connecting part at an opposed end to the first end.

4. The trocar of claim 3 in which the open side extends through the needle connecting part.

5. The trocar of claim 3 in which the needle connecting part is a collar.

6. The trocar of claim 1 in combination with a needle.

7. The trocar of claim 6, in combination with the needle, in which the needle has a slot for receiving the trocar.

8. A trocar and needle assembly, comprising:
a needle having a handle;
a trocar having a hollow barrel with an open side extending along the hollow barrel, the hollow barrel terminating at a first end with a tip for placement of the trocar through tissue, the trocar having a needle connecting part at an end opposed to the first end; and
the handle having a connecting part for holding the trocar around the needle.

9. The trocar and needle assembly of claim 8 in which the handle comprising parts that slide relative to each other to disengage the trocar from the handle.

10. The trocar and needle assembly of claim 8 in which the tip is rounded.

11. The trocar and needle assembly of claim 8 in which the needle connecting part is a collar.

12. A method of placing a drainage tube in an eye, comprising;
placing a trocar on the eye, where the trocar has a trough shape and a base, with the trough opening in a direction that allows access for insertion of a drainage tube;
placing a drainage tube in the trocar ready for insertion of the tube;
turning the trocar to allow removal of the trocar; and
removing the trocar leaving the tube in place on the eye.
